(19) United States
(12) Patent Application Publication
(10) Pub. No.: US 2015/0150460 A1
(43) Pub. Date: Jun. 4, 2015

(54) Methods And Systems For Intraoperative Tumor Margin Assessment In Surgical Cavities And Resected Tissue Specimens

(71) Applicant: The Trustees of Dartmouth College, Hanover, NH (US)

(72) Inventors: Venkataramanan Krishnaswamy, Lebanon, NH (US); Brian William Pogue, Hanover, NH (US); Ashley Marie Laughney, Whitingham, VT (US); Keith Paulsen, Hanover, NH (US)

(21) Appl. No.: 14/406,487

(22) PCT Filed: Jun. 7, 2013
(86) PCT No.: PCT/US13/44803

§ 371 Date: Dec. 8, 2014

Related U.S. Application Data
(60) Provisional application No. 61/656,823, filed on Jun. 7, 2012.

(51) Int. Cl. A61B 5/00; A61B 1/06; A61B 1/07

(52) U.S. Cl. CPC A61B 5/0075; A61B 5/7264; A61B 5/0084; A61B 1/07; A61B 5/0071; A61B 1/0646; A61B 5/7282; A61B 5/4836

(57) ABSTRACT

A tissue classifying system uses central illumination while detecting scattered light received from one or more rings surrounding the central illumination. A broadband illuminator is used. Received light couples to a spectrographic detection system that provides data to a processor with machine readable instructions for determining a classification of a type of tissue illuminated by the system. A scanner is used to generate a map of tissue classification for use by a surgeon who may remove additional tissue from a surgical wound to ensure complete treatment. Embodiments include a scanner that maps tissue classification across tissue, and a scanner coupled to a coherent optical bundle that may be placed in contact with tissue along boundaries of an operative wound. Other embodiments are adapted to scan tissue for fluorescent emissions and/or polarization shifts between incident and scattered light.

### RELATED APPLICATIONS

The present application claims priority to U.S. Patent Application No. 61/656,823, filed Jun. 7, 2012, the disclosure of which is incorporated herein by reference.

### FEDERAL RIGHTS

The work described herein has received funding under National Cancer Institute, National Institutes of Health grant numbers P01CA80139 and P01CA84203. The United States government has certain rights in the invention.

### FIELD

The present application relates to the field of automated, optical, devices, for classifying mammalian and human tissue types. In particular, the device described permits rapid assessment of surgical margins for presence of cancerous tissue.

### BACKGROUND

Cancer, including breast cancer, is an increasingly common disease and, all too often, a common cause of death in the United States and many other countries.

It is known that patient survival can be reduced if malignant tissue is left in operative sites; in treating cancer surgically, it is generally considered desirable to remove as much as possible, or all, diseased tissue or tumor from a patient in order to provide a cure. Many such operations involve removing considerable adjacent normal tissue along with the tumor to ensure that all possible tumor is removed. It is also true that removal of excessive normal, or stroma, tissue is undesirable as it may cause loss of function, poor cosmetic results, edema, pain and morbidity.

Malignant tumors are often not encapsulated or clearly demarcated; the boundary between tumor and adjacent normal tissue may be uneven with projections and filaments of tumor extending into surrounding normal tissue. Since complete tumor removal is desired, and tumors often have ill-defined boundaries, a surgeon will often attempt to excise the tumor together with a surrounding narrow margin of stroma that may contain projections and filaments of tumor. Under typical operative conditions boundaries between tumor, especially narrow but invasive extensions of tumor, and stroma is not always apparent to the unaided surgeon's eye.

After initial removal of a tumor, it is desirable to inspect boundaries of the surgical cavity to ensure all tumor has been removed; if remaining portions of tumor are detected, additional tissue may be removed to ensure complete tumor removal. Similarly, it is desirable to inspect the removed tumor and its surrounding margin to verify adequate margin by verifying that tumor does not reach outer boundaries of the removed tissue.

Conventionally, boundaries of the surgical cavity have been inspected visually by a surgeon. A surgical microscope may be used for this inspection, but small projections and filaments of tumor may escape detection because tumor tissue often superficially resembles normal tissues of the organ within which the tumor first arose. Further, removed tissue may be sectioned and inspected by a pathologist to ensure that the surrounding margin of normal tissue is of adequate thickness such that it is unlikely that filaments and projections of tumor tissue have been left in the patient; this has been done intraoperatively using frozen sections and followed up with microscopic evaluation of stained sections. Evaluation of stained sections may include both common stains and tumor-specific stains for providing good contrast between tumor and stroma.

Stained sections are typically not available until days after completion of the surgery because common techniques require dehydration of specimens, replacing water with paraffin. Further, it is generally not practical to examine frozen or stained sections of organ portions remaining in a patient after tumor resection or of the surgical cavity boundaries.

The current standard of care requires that the margin of stroma surrounding the tumor be examined to verify that no tumor exists within a boundary-layer of the margin in order to verify that all tumor has been removed. For example, for some breast cancers, if tumor is found within a millimeter of the surface of removed margin tissue, it is presumed that tumor may extend into surrounding, un-removed, tissue—requiring additional tissue removal.

Removal of additional tissue days after initial surgery, or reoperation, can pose difficulties, as the patient may require recall to the hospital or surgical center, requires re-anesthetization, and the already-healing wound must then be reopened; causing additional mental and physical trauma to the patient. Some researchers have stated that reoperation may be advised for as many as 40% of surgically-treated breast cancer patients.

In order to prevent reoperation, it is desirable to provide improved apparatus and methods for assessing removed tissue margins, and surgical cavity boundaries, usable at the time of initial surgery to ensure tumors are removed with adequate margins and reduce the likelihood of reoperation.

### SUMMARY

A tissue classifying system uses central illumination while detecting scattered light using a ring of receive optical fibers having ends formed into a planar array and surrounding a central source fiber, A broadband illuminator is coupled to the source fiber. The receive fibers couple to a spectrographic detection system that provides data to a processor with machine readable instructions for determining a classification of a type of tissue illuminated by the source fiber. Embodiments include a handheld probe, a scanner that maps tissue classification across tissue, and a scanner coupled to a coherent optical bundle that may be used to directly scan tissue along boundaries of an operative wound, and embodiments having additional rings of receive fibers.

In a particular embodiment, a tissue classifying system uses central illumination while detecting scattered light using a ring of receive optical fibers having ends formed into a planar array and surrounding a central source fiber, a broadband illuminator is coupled to the source fiber. The receive fibers couple to a spectrographic detection system that provides data to a processor with machine readable instructions for determining a classification of a type of tissue illuminated by the source fiber. The system has a scanning device for scanning the light of the source fiber across tissue, and the processor has instructions to generate a map showing tissue type across the surface of the tissue.

In an alternative embodiment, a method of classifying a type of tissue requires illuminating a classification location on the tissue with a broad-spectrum light, capturing spectra from at least an inner and an outer ring of tissue surrounding the illuminated location, and using the captured spectra in an automatic classifier to determine a tissue type. In a particular variation, the method also includes determining textural parameters from an array of locations surrounding the classification location, and using those textural parameters during classification.

In another alternative embodiment, A central-illumination scattering-based tissue-classifying system designated C including: a coherent bundle of optical fibers, the bundle having a first end and a second end, the second end configured for placement against tissue; a broadband illuminator coupled to illuminate a first region on the first end of the bundle; optics configured to collect light received from a first annular region surrounding the first region of the bundle into at least a first channel of a spectrographic detection system; apparatus configured to scan the first region and the first annular region across the first end of the bundle; a processor coupled to receive data from the spectrographic detection system and having machine readable instructions for determining a classification of a type of tissue illuminated by light from the second end of the bundle based upon spectra of light scattered by the tissue, and to provide a representation of tissue type distribution across the tissue.

### BRIEF DESCRIPTION OF THE FIGURES

FIG. 1 is a block diagram of a system for automatically identifying tumor tissue and for providing guidance to a surgeon during surgery.

FIG. 2 is a block diagram of an alternative embodiment of an imaging head for the system.

FIG. 3 is a flowchart of a method of determining a training database for a kNN-type classifier for identifying tumor tissue.

FIG. 4 is a flowchart of a method of determining types of tissue in a field of view and providing guidance to a surgeon during surgery.

FIG. 5 is a block diagram of an enhanced embodiment of a system for automatically identifying tumor tissue and for providing guidance to a surgeon.

FIG. 6 is a block diagram of an alternative embodiment of a scan head of the embodiment of FIG. 5, wherein a circular mirror is used in place of the annular mirror of FIG. 5.

FIG. 7 is a schematic illustration of an embodiment with central illumination and annular detection.

FIG. 8 is a schematic illustration of fibers at a focal plane of lens 505 of the embodiment of FIG. 7.

FIG. 9 is a schematic illustration of a multichannel spectrographic detector.

FIG. 10 is a schematic illustration of the scanning system used with a coherent fiber bundle for inspection of borders of a surgical cavity.

FIG. 11 is a schematic illustration of a handheld probe, or a mechanically-scanned single-point probe, suitable for classifying tissue at individual points along boundaries of an operative wound with axial illumination and a multichannel spectrographic detector.

FIG. 12 is an approximate flowchart illustrating a method of mapping tissue classifications of resected tumor margins, or of portion of a surgical cavity surrounding where tissue has been resected.

FIG. 13 is a block diagram of an alternative embodiment having a polarizing beam-splitter for enhanced contrast and tissue specificity.

### DETAILED DESCRIPTION OF THE EMBODIMENTS

Localized reflectance measurements of tissue are dependent on local microstructure of the tissue. Since microstructure of tumor tissue often differs in some ways from that of normal tissue in the same organ, localized reflectance measurement of tumor tissue may produce reflectance readings that differ from those obtained from localized reflectance measurements of normal tissue in the same organ.

In a study, reflectance spectrographic measurements of necrotic tumor tissue were shown to vary as much as 50% from measurements of normal tissue, and spectroscopic reflectance measurements of rapidly dividing malignant tumor tissue were shown to vary by as much as 25% from measurements of normal tissue of the type from which the tumor tissue arose.

Most normal organs have at least some degree of heterogeneity, often including such structures as ducts and vessels as well as organ stroma, and organs may be in close proximity to other structures such as nerves. The normal organ stroma of many organs, including kidneys, adrenals, and brains, also varies from one part of the organ to another. The net effect is that there are often multiple normal tissue types in an organ.

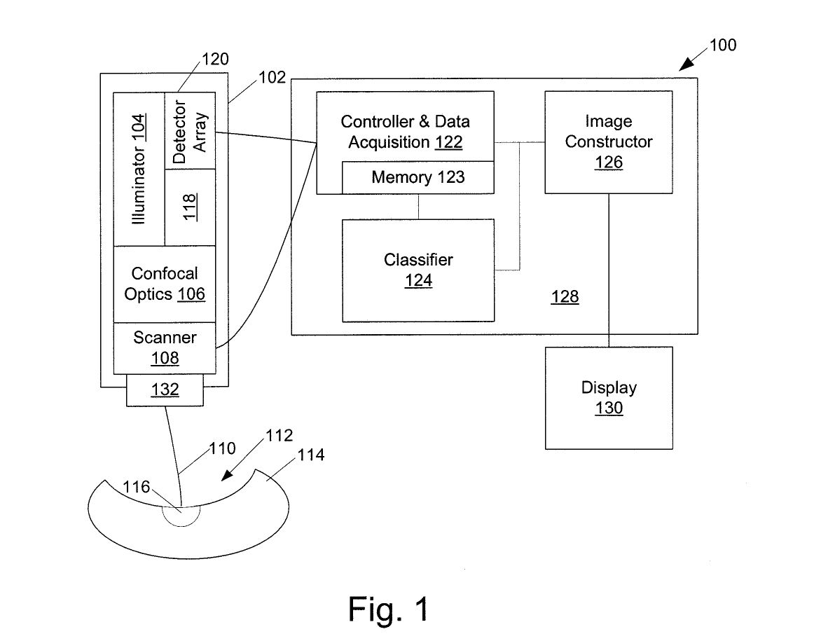

An instrument 100 for assisting a surgeon in surgery is illustrated in FIG. 1. The instrument has an imaging head 102 that is adapted for being positioned over an operative site during surgery. Imaging head has an illuminator subsystem 104 that provides a beam of light through confocal optics 106 to scanner 108. Scanner 108 scans the beam of light 110 through objective lens system 132 onto an operative cavity 112 in an organ 114. A tumor portion 116 may be present in a field of view over which scanner 108 directs beam 110 in cavity 112 in organ 114. Light scattered from the organ 114 and tumor 116 is received through scanner 108 and confocal optics 106 into a spectral separator 118 into a photodetector array 120. Spectral separator 118 is typically selected from a prism or a diffraction grating, and photodetector array 120 is typically selected from a charge-coupled-device (CCD), or CMOS sensor having an array of detector elements, or may be multiple photomultiplier tubes or other photodetector elements as known in the art of photosensors.

Incident light scattered by tissue may be scattered singly, twice, thrice, or more times before it leaves the tissue. Incident light may also be specularly reflected from the tissue surface, with such reflections returning directly from tissue surface to the scanner.

It has been found that light that is specularly reflected from tissue surface carries little information of tissue type. Further, light scattered many times may be affected by deep-lying tissue, as well as tissue laterally displaced from where the light arrived on the tissue; light scattered only a few times tends to carry more information about tissue types near the tissue surface. Further, light scattered many times also increases its chance of being absorbed by tissue constituents, including oxygenated and de-oxygenated hemoglobin. In these cases, the detected signal is sensitive to both absorption and scattering properties of tissue, and complex modeling and additional independent measurements are often needed to decouple the effects of absorption and scatter, to estimate the relative contributions. Typically, light scattering signals are sensitive to changes in tissue ultrastructure and morphology, while absorption signals are sensitive to functional changes in tissue, such as hemoglobin concentration, oxygenation etc.

Signals from photodetector array 120 incorporate a spectrum of received scattered light for each spot illuminated as scanner 108 raster-scans a field of view on organ 114 and tumor 116, and are passed to a controller and data acquisition subsystem 122 for digitization and parameterization; scanner 108 operates under direction of and is synchronized to controller and data acquisition subsystem 122.

Digitized and parameterized signals from photodetector array 120 are passed to a classifier 124 that determines a tissue type of tissue for each location illuminated by beam 110 in organ 114 or tumor 116, and an image is constructed by image constructor and recorder 126. In an embodiment, conventional optical images of the operative site and images of maps of determined tissue types are constructed. Controller and data acquisition subsystem 122, classifier 124, and image constructor 126 collectively form an image processing system 128, which may incorporate one or more processors and memory subsystems. Constructed images, including both conventional optical images and maps of tissue types are displayed on a display device 130 for viewing by a surgeon.

In an alternative embodiment, a diverter or beam-splitter (not shown in FIG. 1) as known in the art of surgical microscopes, may be provided to permit direct viewing by a surgeon through eyepieces (not shown). In an alternative embodiment, digitization may be performed at detector array 120 instead of controller and data acquisition system 122.

In a particular embodiment, illuminator 104 is a tungsten halogen white light source remotely located from imaging head 102, but coupled through an optical fiber into imaging head 102. In this embodiment, the beam 110 illuminates an illuminated a spot of less than one hundred microns diameter on the surface of tumor 116 and organ 114 and contains wavelengths ranging from four hundred fifty to eight hundred nanometers. The spot size of less than one hundred microns diameter was chosen to avoid excessive contributions to the received light from multiple scatter in the organ 114 and tumor 116 tissue; with small spot sizes of under one hundred microns diameter a majority of received light is singly scattered thereby permitting the system to derive tissue-type information primarily from light scattered only once or a few times.

In this embodiment, confocal optics 106 incorporates a beamsplitter for separating incident light of the beam from light, hereinafter received light, scattered and reflected by organ 114 and tumor 116. The received light is focused on a one hundred micron diameter optical fiber to serve as a detection pinhole, and light propagated through the fiber is spectrally separated by a diffraction grating and received by a CCD photodetector to provide a digitized spectrum of the received light for each scanned spot.

The optical system, including confocal optics 106, scanner 108, and objective 132 has a depth of focus such that the effective field of view in the organ 114 and tumor 116 is limited to a few hundred microns.

Scanner 108 may be a galvanometer scanner or a rotating mirror scanner as known in the art of scanning optics. The scanner 108 moves the spot illuminated by beam 110 over an entire region of interest of the organ 114 and tumor 116 to form a scanned image. Spectra from many spot locations scanned on the surface of organ 114 and tumor 116 in a field of view are stored in a memory 123 as pixel spectra of an image.

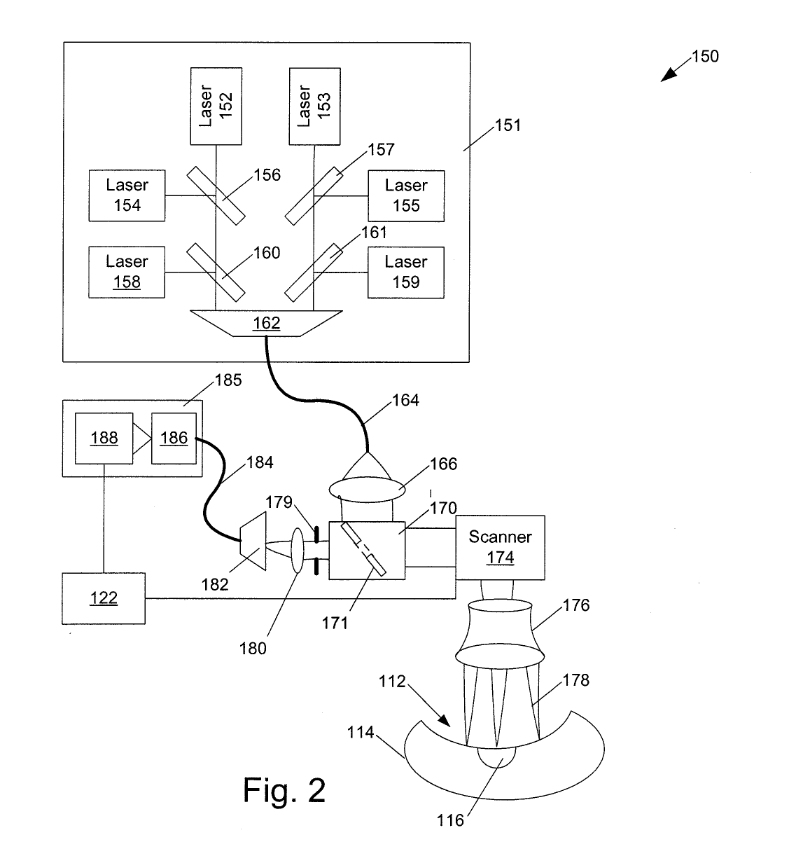

In an alternative image head embodiment 150, illustrated in FIG. 2, illuminator 151 has several lasers. In a particular embodiment there are six lasers 152, 153, 154, 155, 158, and 159. Each laser operates at a different wavelength; in this particular embodiment wavelengths of 405, 473, 532, 643, 660, and 690 nanometers are used. In variations of this embodiment, additional lasers at other or additional wavelengths are used. Beams from these lasers 152, 153, 154, 155, 158, and 159 are combined by dichroic mirrors 156, 157, 160, 161 and combined and coupled into an optical fiber 164 by coupler 162. Light from illuminator 151 is therefore composite light from a plurality of monochromatic laser light sources.

Light from illuminator 151 is directed by lens 166 into separator 170 containing a mirror 171. Light from illuminator 151 leaves separator 170 as an annular ring and is scanned by scanner 174. Scanner 174 may incorporate a rotating mirror scanner, an X-Y galvanometer, a combination of a rotating mirror in one axis and galvanometer in a second axis, or a mirror independently steerable in two axes.

Light from scanner 174 is directed through lens 176 onto the organ 114 and tumor 116 in operative cavity 112. Light, such as light 178 scattered by the organ 114 and tumor 116 is collected through lens 176 and scanner 174 into separator 170 in the center of the annular illumination. In this embodiment, lens 176 is a telecentric, color-corrected, f-theta scan lens, in one particular embodiment this lens has a focal length of approximately eight centimeters, and is capable of scanning a two by two centimeter field. Light in the center of the beam is passed by separator 170 through an aperture 179, a lens 180 and a coupler 182 into a second optical fiber 184. Aperture 179 may be an effective aperture formed by one or more components of separator 170 or may be a separate component.

Optical fiber 184 directs the light into a spectrally sensitive detector 185, or spectrophotometer, having a dispersive device 186, such as a prism or diffraction grating, and a photosensor array 188. Photosensor array 188 may incorporate an array of charge coupled device (CCD) photodetector elements, complementary metal oxide semiconductor (CMOS) photodetector elements, P-Intrinsic-N (PIN) diode photodetector elements, or other photodetector elements as known in the art of photosensors. Signals from photosensor array 188 enter the controller and data acquisition system 122 of image processing system 128 (FIG. 1), and scanner 174 operates under control of controller and data acquisition system 122. Remaining elements of image processing system 128, as well as display 130 are similar to those of FIG. 1 and will not be separately described here.

In the embodiment of FIG. 21A, illumination light from annular mirror 171 forms a hollow cone, and received light is received from within the center of the illumination cone. This arrangement helps to reject light from specular reflection at surfaces of the organ 114 and tumor 116. This arrangement may be achieved by using a ring-shaped mirror 171 in separator 170, or in another variation by swapping the illumination entrance and spectrometer exit ports of separator 170 and using a small discoidal mirror in separator 170.

In an alternative embodiment, similar to that of FIG. 2, lasers having wavelengths from six hundred to nine hundred nanometers are used.

Once digitized, the pixel spectra are corrected for spectral response of the instrument 100. The corrected spectra are parameterized for hemoglobin concentration and degree of oxygenation by curve-fitting to known spectra of oxygenated HbO and deoxygenated Hb hemoglobin. The spectra are also parameterized for received brightness in the six hundred ten to seven hundred eighty five nanometer portion of the spectrum, which is a group of wavelengths where hemoglobin absorption is of less significance than at shorter wavelengths. The Hb and HbO parameters are used for correction of the scatter parameters.

The scattered reflectance and average scattered power at each of several wavelengths in the obtained spectra are calculated using the empirical equation:

I_{R}=Aλ^{-b}exp(−kc(d(HbO(λ))+(1−d)Hb(λ)))

where λ is wavelength, A is the scattered amplitude, b is the scattering power, c is proportional to the concentration of whole blood, k is the path length of incident light in the organ 114 and tumor 116 tissue, and d is the hemoglobin oxygen saturation fraction. In the embodiment of FIG. 2, the wavelengths of each laser are used in the equation. An average scattered reflectance I_{RAVG} is determined by integrating I_{R} over the wavelength range from the six hundred ten to seven hundred eighty five nanometers to provide an average reflectance.

The extracted reflectance and scatter power, and average scatter parameters are then unity normalized according to the mean of all parameters of the same type throughout the scanned image, and dynamic range compensation is performed, before these parameters are used by classifier 124.

There are many different organs found in a typical human body. Each organ has one or several normal tissue types that have scatter parameters that in some cases may differ considerably from scatter parameters of normal tissue types of a different organ. Further, abnormal tissue, including tissue of a tumor, in one organ may resemble normal tissue of a different organ—for example a teratoma on an ovary may contain tissue that resembles teeth, bone, or hair. Metastatic tumors are particularly likely to resemble tissue of a different organ. For this reason, in an embodiment the classifier is a K-Nearest Neighbors (kNN) classifier 124 that is trained with a separate training database for each different organ type that may be of interest in expected surgical patients. For example, there may be separate training databases for prostates containing scatter information and classification information for normal prostate tissues and prostate tumors, another for breast containing scatter information for normal breast and breast tumors, another for pancreas containing scatter information for normal pancreatic tissues and pancreatic tumors, and another for brain containing scatter information for normal brain tissues as well as brain tumors including gliomas.

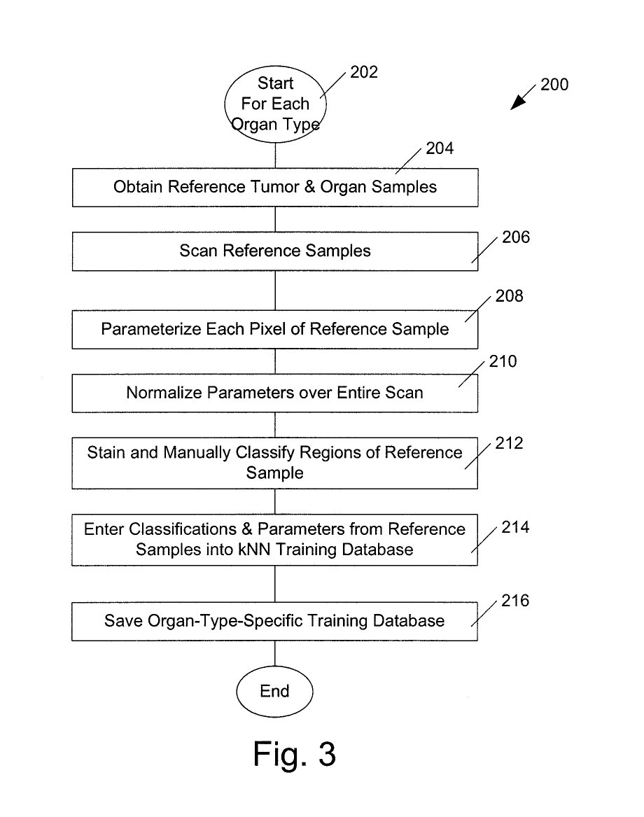

The kNN classifier 124 is therefore trained according to the procedure 200 illustrated in FIG. 3 for each organ type of interest in a group of expected surgical patients. Samples of organs with tumors of tumor types similar to those of expected surgical patients are obtained 204 as reference samples. The reference samples are scanned 206 with an optical system 102 like that previously discussed with reference to FIG. 1 to generate pixels of a reference image. The reference image is parameterized 208 and normalized 210 in the same manner as pixels of images to be obtained during surgery and as discussed above. The reference samples are then fixed and paraffin encapsulated. A surface slice of each sample is stained with hematoxylin and eosin as known in the art of Pathology, and subjected to inspection by one or more pathologists. The pathologists identify particular regions of interest according to tissue types seen in the samples 212. The tissue is classified according to tissue types of interest during cancer surgery, including normal organ capsule and stroma, necrotic tumor tissue, rapidly dividing tumor tissue, fibrotic regions, vessels, and other tissue types that are selected according to the tumor type and organ type.

The parameters for pixels in regions of interest 214 are entered with the pathologist's classification for the region into the training database for the kNN classifier 124. After the reference samples for organs of this type are processed, an organ-specific database is saved 216 for use in surgery.

In a study, similar hardware having a mechanical scanning arrangement instead of a mirror scanner but capable of determining the same reflectance, Hb, and HbO2 parameters, was used to scan samples of pancreatic and prostate tumors grown in rodents. Once scanned to determine a training parameter set corresponding to in-vivo tissue parameters, a surface slice of each sample was encapsulated, fixed, stained with hematoxylin and eosin as known in the art of Pathology, and subjected to inspection by a pathologist. The pathologist identified particular regions of interest in the sections according to tissue types seen in the sections. These included:
epithelial cells with low nucleus to cytoplasm ratio (these are believed to be mature tumor cells);
epithelial cells with high nucleus to cytoplasm ratio (these are believed to be proliferating tumor cells);
fibrotic regions of early fibrosis;
fibrotic regions of intermediate fibrosis;
fibrotic regions of mature fibrosis;
regions of exudative necrosis; and
regions of focal necrosis.

It should be noted that the tumor type being classified in this experiment was a tumor of an epithelial cell type. The parameters for a subset of pixels of each region of interest, together with the pathologist's classifications were used to train a kNN (k-Nearest-Neighbors) classifier.

Performance of the kNN classifier against unknown pixel data was verified by classifying a different subset of pixels of the same regions with the kNN classifier with a high degree of consistency.

The kNN classifier 124 operates by finding a distance D between a sample set of parameters s corresponding to a particular pixel P and parameter sets in its training database. For example, in an embodiment, at each particular pixel P, if there are M entries in the training database, M distances are calculated from measurements according to the formula

D(pₛ,pₙ)={square root over ((Aₛ−Aₙ)²+(bₛ−bₙ)²+I_{avgs}−I_{avgn})²)} for n=1 to M.

The scanned pixel P is classified according to the classification assigned in the training database to parameter sets giving the smallest distance D. In alternative embodiments, distance D is computed using other statistical distances instead of the formula above, such as those given by Mahalanobis, Bhattacharyya, or other distance formulas as known in the art of statistics. It is expected that a kNN classifier using the Mahalanobis distance formula may provide more accurate classification than the Euclidean distance formula.

In a particular embodiment, each pixel spectra is obtained by measuring intensity at six discrete wavelengths in the 400-700 nanometer range. In alternative embodiments, additional wavelengths are used.

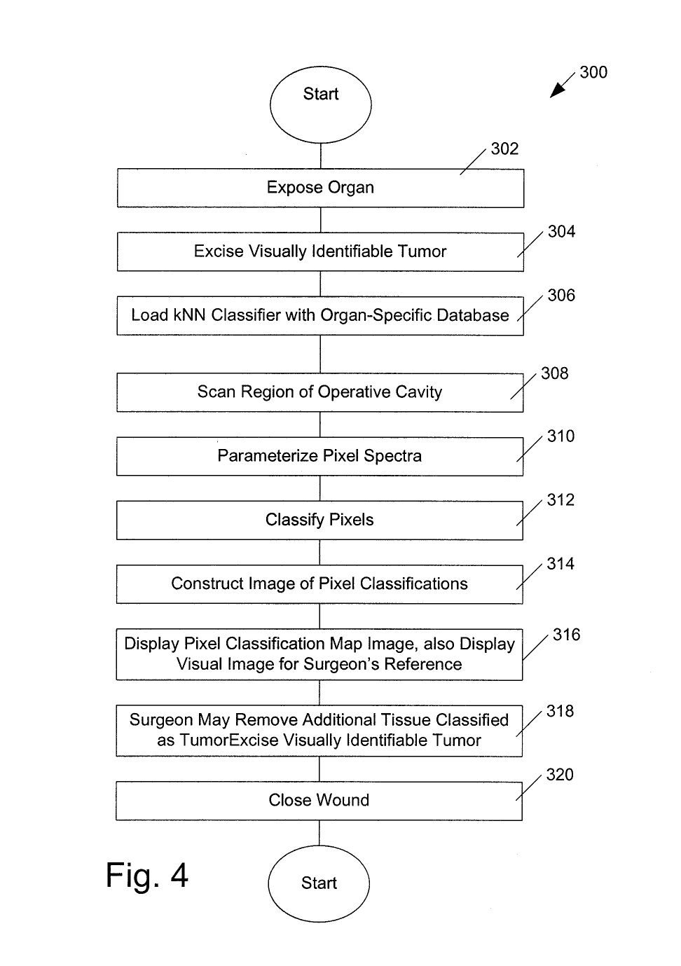

In the surgical procedure 300 illustrated in FIG. 4, the organ of interest is exposed 302 by the surgeon. The surgeon then excises 304 those portions of tumor that are visually identifiable as such as known in the art of surgery. Meanwhile, the kNN classifier 124 is loaded 306 with an appropriate organ-specific database saved at the end of the reference classification procedure of FIG. 3.

A region of interest in the operative cavity is scanned 308 by optical system 102, an array of pixel spectra obtained is parameterized 310, the pixels are classified 312 by classifier 124, and a map image of the classifications is constructed 314. The classifier classifies the tissue at least as tumor tissue and normal organ tissue, in an alternative embodiment the classifier classifies the tissue as normal organ tissue, rapidly proliferating tumor tissue, mature tumor tissue, fibrotic tissue, and necrotic tissue. In an embodiment, the map image is color encoded pink for mature tumor tissue, red for rapidly proliferating tumor tissue, and blue for normal organ tissue. In alternative embodiments, other color schemes may be used. The classification map is displayed 316 to the surgeon. The surgeon may also view a corresponding raw visual image to orient the map in the region of interest. The surgeon may then excise 318 additional tumor, and repeat steps 308-318 as needed before closing 320 the wound.

In an alternative embodiment, in addition to the three scatter-related parameters heretofore discussed with reference to kNN classifier 124, additional parameters are defined for each pixel both during training of the classifier and intraoperatively. These additional parameters include statistics such as mean, standard deviation, a skew measure, and a kurtosis measure, and in alternative embodiments include additional parameters derived from texture features such as contrast, energy, entropy, correlation, sum average, sum entropy, difference average, difference entropy and homogeneity, of reflectance in a window centered upon the pixel being classified. These parameters are collectively referred to as statistical parameters. Adding these parameters to the parameters used for classification by the kNN classifier 124 appears to improve accuracy of the resulting map of tissue classifications. In this classifier, an alternative formula, having weights for each parameter, for calculating distance was used, according to the Bhattacharya statistical distance. In this measure, the difference in a scattering parameter p, with p=1, 2, . . . , 15, between two tissue subtypes, i and j, is given by:

$${J}_{ij}^{p} = \frac{1}{4} {{( {μ}_{j} - {μ}_{i} )}^{T} [ {Σ}_{i} + {Σ}_{j} ]}^{- 1} ( {μ}_{j} - {μ}_{i} ) + \frac{1}{2} ln ( \frac{\vert {Σ}_{i} + {Σ}_{j} \vert}{2 {( \vert {Σ}_{i} \vert \cdot \vert {Σ}_{j} \vert )}^{\frac{1}{2}}} )$$

where μᵢ and Σᵢ are the mean and the variance matrix of p for tissue sub-type i. Further, Jᵢⱼ is the distance between sub-types i and j. For smaller window sizes, which means that mostly vicinity regions will be within the same tissue sub-type, the mean scattering power is always selected as the most discriminating feature.

In this embodiment, experiments have been performed using window sizes of from four by four pixels to twelve by twelve pixels centered upon the pixel being classified. This classifier gave classifications that more closely matched those given by the pathologist than those provided by using only scatter parameters in the classifier.

In an alternative embodiment 400 having enhanced capabilities, a different light source 401 is used which differs from the light source 151 illustrated in the embodiments of FIG. 2. Light source 401 has a broad spectrum, or white-light-producing element that provides radiation across a wide selection of wavelengths ranging from the visible through the infrared. In an embodiment, the light producing element is a supercontinuum laser 402 having significant output ranging from wavelengths of nearly four hundred nanometers to greater than two thousand nanometers. Supercontinuum lasers covering this broad spectral range are available from NKT Photonics, Birkerod, Denmark, although other sources may be used.

Light from laser 402 is passed through a filter 404 that passes a wavelength range of particular interest for determining scatter signatures of normal and tumor cells, while blocking light at the infrared end of the spectrum that may cause undue heating of components and use of which would require detectors made of exotic materials other than silicon. In an embodiment, filter 404 passes a range of radiation from 400 to 750 nanometers, in an alternative embodiment laser 402 emits light of wavelengths 400 nanometers and longer, while filter 404 is a high-pass filter that passes wavelengths shorter than 750 nanometers.

Light passed by bandpass filter 404 is divided into two beams by a beamsplitter 406. One beam from beamsplitter 406 passes to a high speed, electronically operated, optical beam switching device 410. A second beam from beamsplitter 406 passes through a tunable filter 408 and then to switching device 410. In an embodiment, tunable filter 408 is an acousto-optic tunable filter; in an alternative embodiment tunable filter 408 is a rotary filter having several bandpass elements having different center frequencies and which rotates under computer control to change wavelengths of light passing through filter 408. An alternative embodiment filter 408 is a liquid crystal tunable.

Computer-controlled optical switch 410 selects light from a desired path from tunable filter 408 or beamsplitter 406, and passes the light to a fiber coupler 412. Fiber coupler 412 couples the light into a source optical fiber 414. In an embodiment, optical fiber 414 is a single mode fiber of about five microns diameter. The entire light source 401 operates under control of a local microcontroller 416.

As with the embodiment of FIG. 2, light from optical fiber 414 passes through a lens 420 into separator 422 containing an annular mirror 424. Light from fiber 414 leaves separator 422 as an annular ring and is scanned by scanner 428. Scanner 428 may incorporate a rotating mirror scanner, an X-Y galvanometer, a combination of a rotating mirror in one axis and galvanometer in a second axis, or a mirror independently steerable in two axes.

Light from scanner 428 is directed through lens 430 onto the organ 114 and tumor 116 tissues in operative cavity 112. The scanner 428 causes the light to scan across an opening or window of probe 426, which in an embodiment is a handheld probe and in an alternative embodiment is a stand-mounted probe, beneath lens 430, this light is illustrated at several scanned beam 432 positions. Light, such as light 432 scattered by the organ 114 and tumor 116 tissues is collected through the same lens 430 and scanner 428 into separator 422, where it passes through an aperture 423. At least some of light 432 is returned to separator 422 in the center of the beam, and passes through another lens 440 and coupler 444 into a receive fiber 442.

In an embodiment, lens 430 is a telecentric, color-corrected, f-theta scan lens, in one particular embodiment this lens has a focal length of eight centimeters, and is capable of scanning a two by two centimeter field. In an embodiment, aperture 423 may be an effective aperture formed by one or more components of separator 422, such as a central hole in mirror 424, or may be a separate component.

Optical fiber 422 directs the light into a spectrally sensitive detector 448, or spectrophotometer, having a dispersive device 450, such as a prism or diffraction grating, and a photosensor array 452. Photosensor array 452 may incorporate an array of charge coupled device (CCD) photodetector elements, complementary metal oxide semiconductor (CMOS) photodetector elements, P-Intrinsic-N (PIN) diode photodetector elements, or other photodetector elements as known in the art of visible and near-infrared-sensitive photosensors. Signals from photosensor array 452 enter the controller and data acquisition system 460 of image processing system 462. Scanner 428, as well as light source 401 through its microcontroller 416 operates under control of controller and data acquisition system 460. Remaining elements of image processing system 462, as well as display 464, are similar to those of image processing system 128 and display 130 of FIG. 1 and will not be separately described here.

In a scattering-based mode of operation, beam switch 410 passes light from filter 404 into fiber coupler 412, and thence to tumor 116. Photosensor array 452 receives and performs spectral analysis of light scattered by tissue of organ 114 and tumor 116, and received through spectrally sensitive detector 448, and processing system 462 uses a kNN classifier as previously discussed to classify tissue as tumor tissue or normal tissue. In an alternative embodiment, the processing system may use another classifying scheme known in the art of computing such as artificial neural networks, and genetic algorithms.

In particular alternative embodiments, the processing system uses an Artificial Neural Network classifier, in another embodiment a Support Vector Machine classifier, in another a Linear Discriminant Analysis classifier, and in another a Spectral Angle Mapper classifier; all as known in the art of computing.

In a fluorescence-based mode of operation, the subject within which organ 114 and tumor 116 tissue lies is administered a fluorescent dye containing either a fluorophore or a prodrug such as 5-aminolevulinic acid (5-ALA) that is metabolized into a fluorophore such as protoporphyrin-IX. Fluorescent dyes may also include a fluorophore-labeled antibody having specific affinity to the tumor 116. With both administered fluorophore or prodrug dyes, fluorophore concentrates in tumor 116 to a greater extent than in normal organ 114. In an alternative, fluorescence, mode of operation, one or the other, or both, of organ 114 and tumor 116 may contain varying concentrations of endogenous fluorophores such as but not limited to naturally occurring protoporphyrin-IX or beta-carotene.

In the fluorescence-based mode of operation, beam switch 410 passes light from tunable filter 408 into fiber coupler 412, and thus into fiber 414 and probe 426. In this mode, tunable filter 408 is configured to pass light of a suitable wavelength for stimulating fluorescence by the fluorophore in organ 114 and tumor 116, while significantly attenuating light at wavelengths of fluorescent light emitted by the fluorophore. Although detector 448 is spectrally sensitive, attenuation of light at wavelengths of fluorescent light by filter 408 increases sensitivity and reduces susceptibility of the system to dirt in the optical paths.

Fluorescent light emitted by fluorophore in organ 114 and tumor 116 is received through lens 430, scanner 428, separator 422, lens 440, coupler 444, fiber 446, into spectrally sensitive detector 448. Spectrally sensitive detector 448 detects the light and passes signals representative of fluorescent light intensity at each pixel of an image of the tissue scanned by scanner 428 as a fluorescence image into image processor 462.

The tunable filter 408 is thereupon changed to other wavelengths and the three specular scatter parameters are determined as discussed above. Image processor 462 thereupon uses the fluorescence intensity and spectrum information as additional information with the three spectral parameters discussed above to classify tissue types in tissue, and displays the tissue classification information to the surgeon. The fluorescence spectrum information is used during classification to allow spectral unmixing of drug and prodrug fluorescence from fluorescence from endogenous fluorophores in tissue. After unmixing, bulk fluorescence is calculated for the given excitation wavelength. Image processor 462 may also present an image of fluorescence to the surgeon.

In an embodiment the ratio of fluorescence intensity to scattered irradiance at the excitation wavelength, which is collected as a part of the scatter mode data, is used as a normalized fluorescence value by the classifier.

In an embodiment, the ratio of fluorescence intensity to scattered irradiance is computed for several different stimulus wavelengths and several different fluorescence wavelengths; in this embodiment these additional ratios are used by the classifier to better distinguish different fluorophores in tumor 116 and organ 114 tissues, and thus to provide improved classification accuracy.

In a fluorescence-only mode of operation of embodiment, fluorescence mode information is used by the classifier without the scattering parameters discussed above; in a synergistic mode of operation both fluorescence mode information, including intensity of fluorescent emissions, and scattering parameters are used by the classifier at each pixel to provide enhanced tissue classification information.

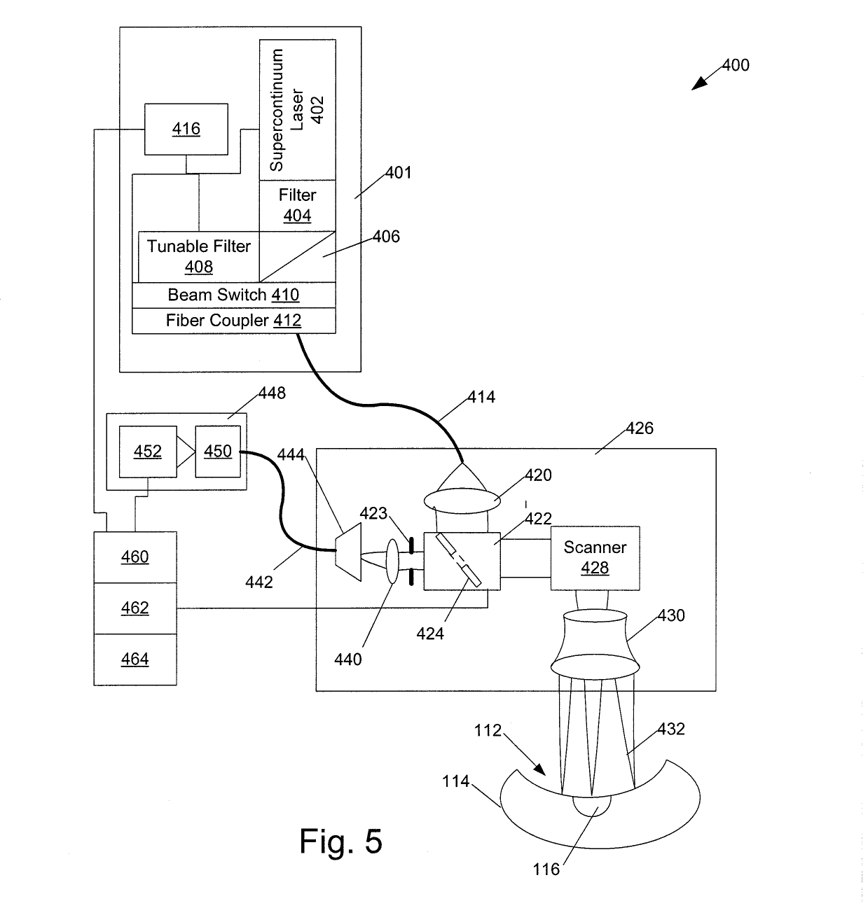

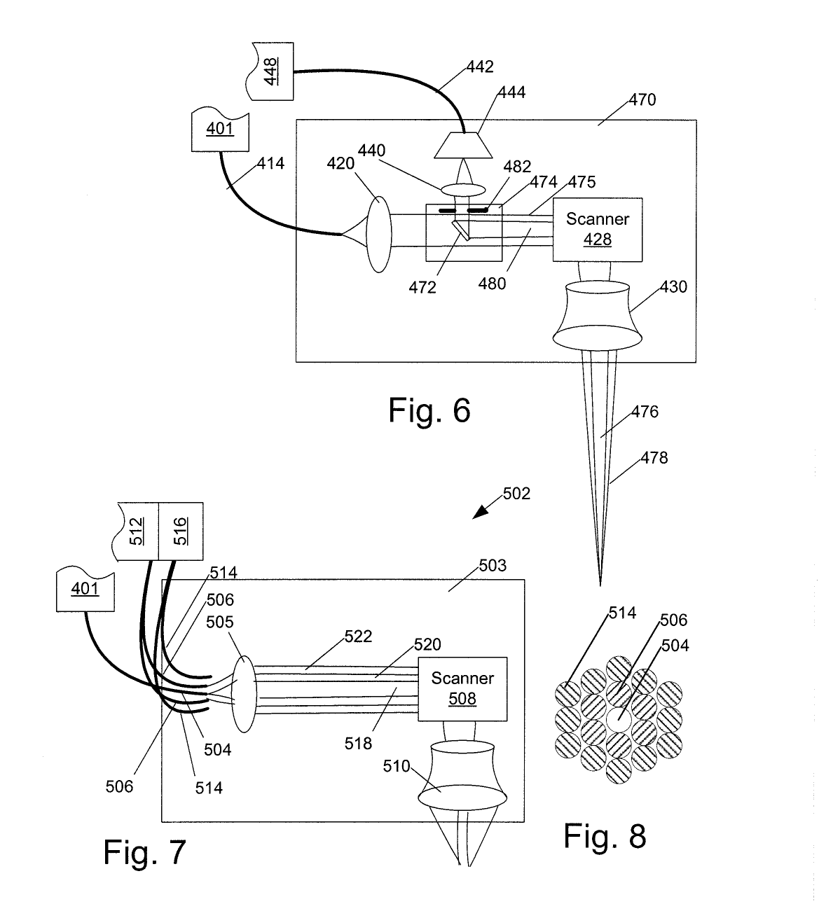

In an alternative embodiment, as illustrated in FIG. 6, resembling that of FIG. 5, a light source 401 identical to that previously discussed with reference to FIG. 5 is used, driving a source optical fiber 414. Similarly, receive optical fiber 442 couples to a spectrally sensitive detector 448 like that previously discussed with reference to FIG. 5. As with FIG. 5, detector 448 feeds an image processing system as previously discussed, in the interest of brevity discussion of the light source, spectrally sensitive detector, and image processing system will not be repeated here.

The embodiment of FIG. 6 differs from the embodiment of FIG. 5 in that probe 470 uses a modified separator 474 having a discoidal mirror 472 instead of the annular mirror 424 of separator 422 of probe 426 of FIG. 5. Source fiber 414 projects light from source 401 through lens 420 around discoidal mirror 472 to form an annular source beam that leaves separator 474 and enters scanner 428; as previously discussed scanner 428 scans this annular illumination 475 through telecentric lens 430 across organ and tumor. Scattered light is received through lens 430 in a central portion 476 of scanned beam 478, and into separator 474 as a received beam 480 contained within annular illumination 475. Discoidal mirror 472 reflects received beam 480 through an aperture 482, which is focused by lens 440 into receive coupler 444 and receive fiber 442 for transmission to the detector

In alternative embodiments, a non-scanning head for the system resembles that of FIG. 5, 6, or 7 except that the scanner 428, and scanning lens 430, are not present. This embodiment is useful as a handheld probe for verifying complete tumor removal by probing suspect areas in a surgical wound.

In another alternative embodiment 502 (FIG. 7), the annular illumination, dark-field illumination with central detection previously discussed is replaced by central-illumination, with annular detection. In this embodiment, light source 401 is coupled to a source end of source fiber 504 and routed to scanning head 503. Another end of source fiber 504 is brought to a focal plane of a lens 505, where it is surrounded by ends of receive fibers 506, the fiber ends organized in a planar array where the source fiber end is central and the receive fiber ends 506 form concentric rings round the source fiber. Light from source fiber 504 passes through lens 505 to a scanner 508, then through scanning lens 510, onto any tissue being inspected. Light scattered from tissue is received through lens 510 and scanner 508, then through lens 505 onto receive fibers 506. In an embodiment, lens 510 is an image-space telecentric lens to ensure the illumination and acceptance cones of light is always perpendicular relative to the tissue surface throughout the scan field. In an embodiment a single ring of receive fibers 506 conducts light to a first detection subsystem 512. In alternative embodiments, receive fiber ends at the focal plane of lens 505 are formed as N rings of fibers, with the case N equals two illustrated in FIG. 8. In this embodiment, each ring of fibers, such as inner ring fibers 506 and outer ring fibers 514 are brought to a separate detection subsystem 512, 516, with light from outer ring fibers going to the second detection subsystem 516. In an embodiment, lens 505 is object-space telecentric to ensure the axis of the effective acceptance cone for the scattered light received by the off-axis collection fibers 506 and 514 is always perpendicular to the face of the fibers.

The size and numerical aperture (NA) of the individual fibers and the separation distance of each ring from the central illuminating fiber are chosen to produce a spot size on tissue that minimizes signal sensitivity to hemoglobin, and allows selective imaging of parameters sensitive to tissue ultrastructure, such as spectral and polarization dependence of scattered light. In a particular embodiment, 10 microns core diameter optical fibers, with an NA of 0.1, and a maximal separation of 200 microns of receive fibers from the illuminating fiber are used. The relatively small distances over which light can interact with tissue and still reach a receive fiber helps permit the system to derive tissue-type information primarily from light scattered only once or a few times.

In an alternative embodiment, a central illuminating fiber of 10 or 50 nanometer core diameter, or of a diameter between 10 and 50 nanometers, is surrounded by concentric rings of receive fibers, the fiber rings having radius of up to two millimeters.

To image absorption and fluorescence features, the size of the fibers, NA and the separation could be increased, at the expense of imaging resolution or maximum field size.

In alternative embodiments, the central fiber is a transmit-receive fiber illuminated through a beam-splitter, which in some embodiments is a polarizing beam splitter, such that the scanning optical system not only collects light received from concentric rings around an illumination spot of tissue on which light from the central fiber is focused, but also simultaneously collects light emitted or reflected back from the illumination spot and collected by the optics in the detection path into the central fiber. Light from tissue collected into the central fiber is directed to a separate channel of the spectrographic detector. This embodiment therefore collects light reflected or emitted from the illumination spot, as well as collecting light emitted from tissue at a predetermined set of radial distances away from that illumination spot (collected by the rings of fibers in the planar array). In a particular embodiment, the collection radial distances are determined by a setting of magnification of the optical system and the fiber separation.

In an embodiment, each detection subsystem 512, 516 of the embodiments illustrated in FIGS. 7 and 8 is a single-channel spectrographic detector. In this system, central light 518 illuminates the tissue, and an inner ring of received light 520 goes to the first detection subsystem 512, and light from an outer ring of received light 522 goes to the second detection subsystem 516. The optical systems, including lens 505 and telecentric lens 510, are configured such that light received from an inner ring of tissue surrounding a point illuminated by central light 518 is received by inner ring of fibers 506, and light from an outer ring of tissue surrounding the inner ring of tissue is received by outer fibers 514.

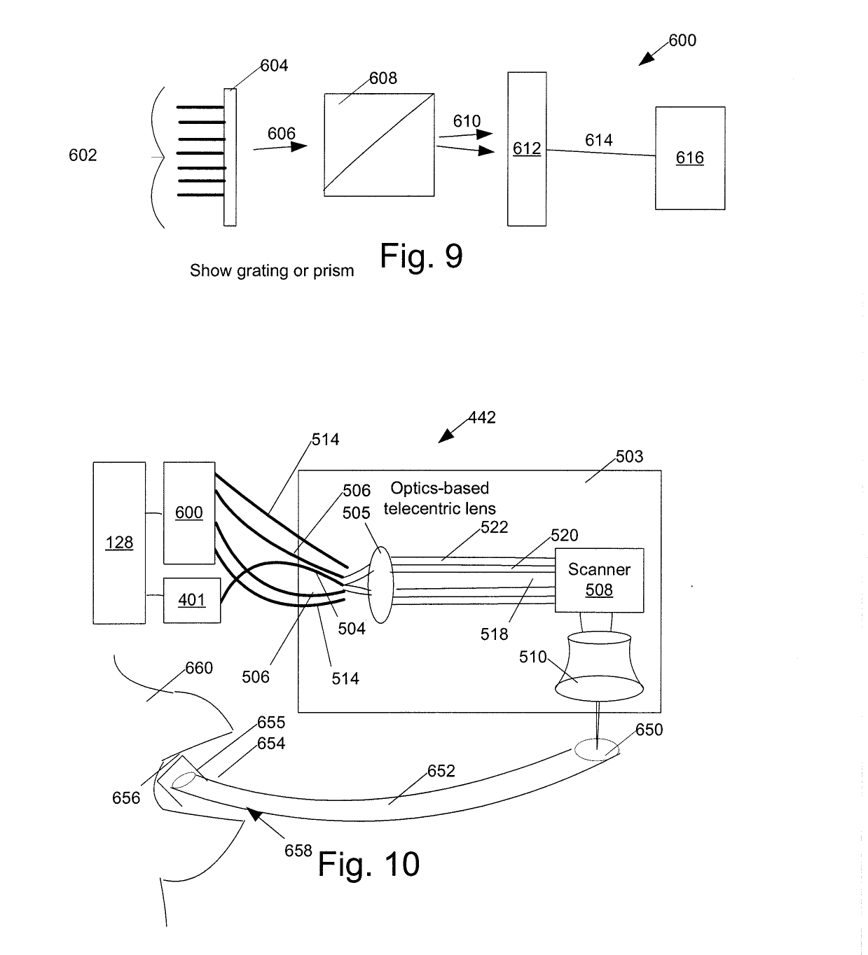

In an alternative multichannel embodiment, a multichannel spectrographic detector 600 (FIG. 9) replaces detection subsystems 512, 516 in the system of FIG. 7. In this embodiment, detector ends of receive optical fibers 514, 506 are formed into a linear array of fibers 602 along a slit 604. Light 606 from the slit passes through a dispersive device 608 such as a prism or a diffraction grid, and light 610 from the dispersive device is received by a rectangular photosensor array 612 such that light from each fiber of fibers 602 illuminates a row of sensors of the photosensor array separate from rows illuminated by each other fiber, and light of a particular wavelength illuminates sensors of each column of the photosensor array; signals 614 from the photosensor array therefore include a spectrum of light received from each fiber independently. A processor 616 is provided for processing these spectra.

The tissue classifying performed by the system described herein is based on light that is scattered by tissue, not light specularly reflected from a surface of tissue. Scanner 508, lens 510, and lens 505 are configured such that light received under normal conditions from a spot on tissue surface that is directly illuminated by light from source fiber 504 is excluded from receive fibers, 506, 514. On occasion, especially where tissue has a somewhat-ragged edge with drops of a liquid adherent to its surface, light is specularly reflected in such manner that it reaches a receive fiber. In the alternative multichannel embodiment, machine readable instructions operable on processor 616 operate to determine channels that receive specularly-reflected light and to exclude spectra from those channels from consideration by the classifier.

Since fluorescent emitted light is at a different wavelength than the stimulus light required to excite its emission, the spectrographic detector can distinguish between light at stimulus and fluorescent wavelengths. In an embodiment, a filter is inserted at light source 401, to block light at the fluorescent emissions wavelength of a particular dye, where the dye has been administered to a patient prior to surgery, and where the dye has been absorbed by part or all of the tissue. Image processing system 128 can then map dye in the tissue by observing light at the fluorescent emissions wavelength.

The scanning head 503 may be difficult to position directly over tissue in a surgical wound, yet it can be desirable to scan for tumor tissue remaining in the bed from which a tumor has been excised as well as on removed surgical samples. Apparatus for scanning tissue at edges of a surgical wound is illustrated in FIG. 10. In this embodiment, a scan head 503 is used. Scan head 503 is similar to that shown in FIG. 7 although here shown coupled to the alternative detector of FIG. 9, and operating under control of, and providing data to, image processing system 128. Scan head 503 is positioned to scan a first end 650 of a flexible, coherent, optical fiber bundle 652. A free end 654 of fiber bundle 652 is adapted such that a surgeon may position the free end 654 adjacent to suspect tissue 656 in surgical cavity 658. In an embodiment, as illustrated in FIG. 10, a “tapered” fiber-optic imaging bundle is used to “magnify” or “demagnify” the effective field imaged on the tissue side, without significant changes to the scanning optics, these embodiments permit use of the system for scanning tissue along sides or bottom of a small surgical cavity that the relatively bulky scanner head 503 cannot fit into with an appropriate viewing angle and viewing distance. In an embodiment, a disposable, thin, clear, polymer cover 655 is provided on the tissue end of fiber bundle 652 for enhanced sterility and to permit rapid clearing of blood and tissue fragments from fiber bundle 652, in other embodiments a disposable fiber bundle 652 is provided. Suspect tissue 656 is typically cut boundaries of tissue where a tumor has been removed, or may be tissue that a surgeon otherwise is uncertain whether removal from the operated organ 660 is indicated. In a particular embodiment, the operated organ 660 is a woman's breast. In various embodiments, coherent fiber bundle 652 may be magnifying, or non-magnifying. In a particular embodiment, second end 654 of coherent bundle 652 is cut at a seven degree angle to minimize reflections as light is coupled into fibers of the bundle while still maintaining an acceptable acceptance cone. In another embodiment, the second end 654 of the coherent bundle 652 is slightly roughened to minimize internal reflections and improve tissue contact. In a particular embodiment, lens 510 is an image space telecentric lens such that the axis of illumination and acceptance cones of light coupled into and received from the fiber bundle 652 is perpendicular to the face of 652 at all field positions.

In an alternative embodiment resembling that of FIG. 10, lens 510 is a lens system that may be magnifying or demagnifying, or in an embodiment a “zoom” optical system that may be adjusted to any of several settings of optical magnification. In this embodiment, a central illuminating spot size of 10 or 50 nanometer diameter is effectively surrounded by concentric rings of receive fibers, the fiber rings of receive fibers having radius of up to two millimeters.

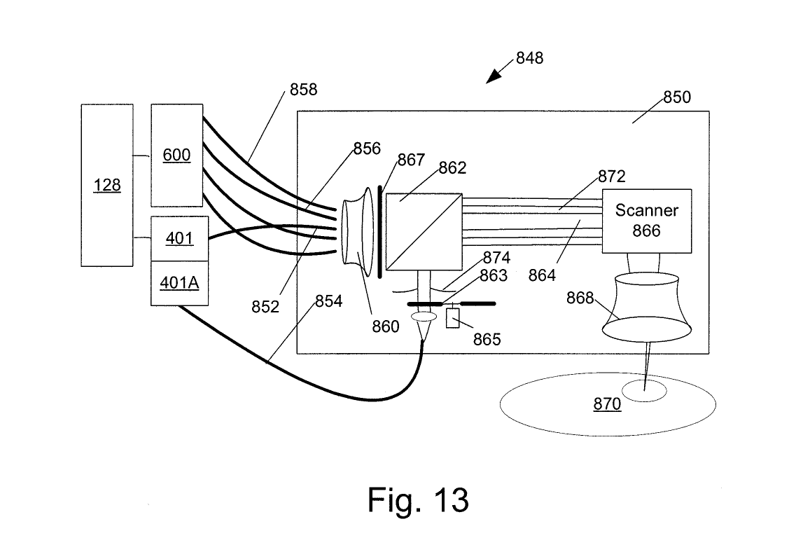

In an alternative embodiment (FIG. 13), a polarizing beamsplitter 862 is used in the scan head 850 to ensure that light from illumination fiber 852 is polarized in a first direction, and only light polarized in a second direction is received by receive fibers 856, 858. See FIG. 13 for a polarizing embodiment. Since light scattered by tissue may be depolarized (due to multiple scattering) or have polarization altered by limited interaction with the tissue, while light that is specularly reflected from tissue retains its original polarization. Since the measurement geometry and sampling spot size is optimized to suppress both specularly reflected light and highly multiply scattered light, this mode mainly measures polarization properties of light scattered once or only a few times, so that by recording light spectra of each pixel at two or more polarization states in this configuration, additional optical parameters sensitive to tissue morphology could be derived to improve tissue-type classification performance.

In a particular embodiment, the apparatus of FIGS. 1-10 is used during surgical removal of ductal carcinoma in situ (DCIS) from a human woman's breast. In alternative embodiments, having different configuration tables in classifier 124, the apparatus is used for removal of tumors from pancreas and brain. During such surgery, the apparatus is used to scan surfaces of a surgical wound, or of a removed surgical specimen, to map tissue type, and the map is presented to a surgeon before the surgical wound is closed. After consulting the map, the surgeon may, when possible, remove additional tissue where tissue classified as of a tumor type remains in the surgical wound, or where the surgical specimen has inadequate surgical margins such that tumor tissue is present at its surface.

In an embodiment, the scanner head 503 scans a 10-centimeter square portion of the tissue with approximately one hundred micron resolution, scanned images are generated in memory of image processing system 128 in an N=one hundred by M=one hundred pixel array, where data stored for each pixel represents spectra received at both at the first and second ring of receive fibers. It is anticipated that other integer values of N and M may be used, including larger arrays.

In a particular embodiment, statistics and textural features are derived from a C-by-D textural classification array textural classification array centered on each pixel of the array that is to be classified by classifier 124. In a particular embodiment, C and D are both chosen as five such that the textural classification array has twenty-five pixels and the classifier can classify all but two rows and two columns of pixels at edges of the N by M scanned array. These second-order statistics are used together with the spectra associated with the pixel to be classified. The particular C=5 and D=5 textural classification array size is chosen because the oxygen diffusion length in tissues is clinically observed to span between one hundred and five hundred micrometers which limits nutrient delivery and the radius of ducts containing proliferating epithelial cells in ductal carcinoma in situ (DCIS). In principle, other array sizes for the C by D textural classification array may be used, especially where scan resolution differs from the one-hundred-micron scan resolution of this embodiment, however N should be an integer at least twice C, and M should be an integer at least twice D.

Reflectance spectra in the waveband that avoids hemoglobin peaks (610:700 nm) behave with a power law dependence (on wavelength); and an empirical approximation to Mie theory was used to describe the relative reflectance spectrum R_{TISSUE} as:

R_{TISSUE},ref(x,y,∀)=A(x,y)∀^{-b(x,y)p} Equation 1

Where A and b are the scattering amplitude and scattering power, respectively. These quantities reflect variations in the size and number density of scattering centers in the volume of tissue probed, which occur on sub-micron and even sub-nanometer length scales. The data-model fitting was log transformed and linear regression was employed to obtain estimates of the scattering amplitude and scattering power relative to Spectralon through direct matrix inversion. Additionally, a measure of average irradiance was calculated by integrating the reflectance spectrum over a waveband that avoids the hemoglobin absorption peaks (610-700 nm).

A gray-level co-occurrence matrix (GLCM) representation of textural features derived from the five by five textural classification array is used, and spatial average, contrast, correlation and homogeneity parameters computed; these parameters are input to the classifier along with spectra obtained with the scanner from the inner and outer receive fiber rings obtained at the pixel-to-be-classified.

A dark-field embodiment of the apparatus, resembling that of FIG. 2 or FIG. 5, including the classifier, was tested on surgical specimens removed from 27 breast cancer patients; after scanning them within one hour of removal from the patient, the specimens were then fixed and processed for conventional hematoxylin-eosin stains and microscopic examination by a pathologist. Portions of tissue scanned included benign pathologies including normal, fibrocystic disease and fibroadenomas. Other portions of tissue scanned included invasive pathologies including DCIS and invasive cancers. The scanned and classified images were co-registered to the hematoxylin-eosin stains and, and then to pathologist reports.

It was found that several scattering parameters derived from the pre-fixing scans, including scattering power, log scattering amplitude, and integrated scattering intensity, were significantly different between tissues of normal, fibrocystic disease, fibroadenomas, DCIS, and invasive cancers, thereby permitting distinguishing tissue type from the scattering parameters. It is expected that the kNN classifier can therefore use these parameters to classify tissue for each pixel.

Results from pair-wise comparisons of the distribution of scattering and texture parameters for some tissue types found in pathological specimens from breast cancer surgery are presented in table 1. Tissue types considered in this table includes Ductal Carcinoma In Situ (DCIS), Normal tissue (NOR), Fibrocystic Disease (FCD), Fibroadenomas (FA), and an Invasive Cancer (INV).

TABLE 1
Pearson's correlation coefficient for pair-wise comparisons between pathologies
per parameter. Underlined values are significant within 95% confidence limits.

| Paired | Scattering | | | | | | Fractal |
|---|---|---|---|---|---|---|---|
| Diagnoses | Power | I_{avg} | Correlation | Contrast | Homogeneity | Euler # | Dimension |
| NOR-INV | 0.0006 | 0.0712 | 0.0150 | 0.0046 | 0.0013 | 0.0018 | 0.0016 |
| NOR-DCIS | 0.0300 | 0.0057 | 6.62E−10 | 0.0171 | 0.0134 | 0.1468 | 0.0007 |
| INV-DCIS | 0.4637 | 0.1553 | 0.0452 | 0.0145 | 0.3038 | 0.2673 | 0.1437 |

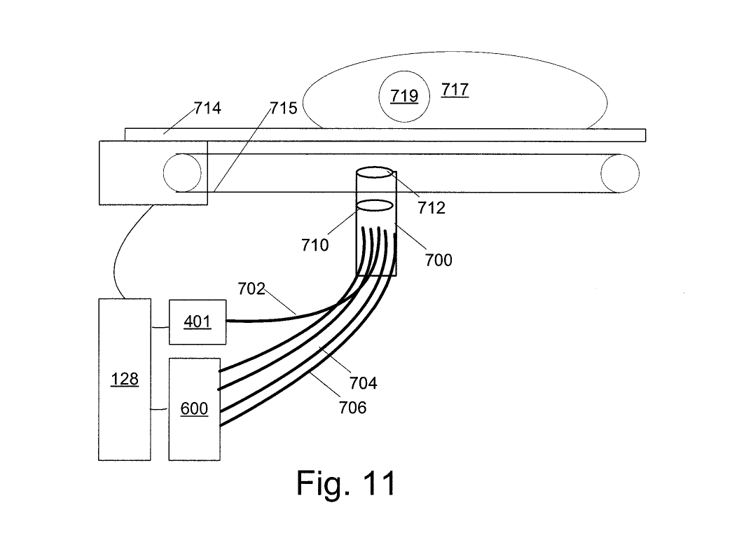

In an alternative embodiment, a handheld probe is provided for probing or classifying suspect areas of walls of an intraoperative surgical cavity. This probe 700 (FIG. 11), has an arrangement of optical fibers with ends of a central illumination fiber 702 surrounded by a ring of inner 704 and a ring of outer 706 receive fibers. Another end of the illumination fiber 702 of probe 700 is coupled to a light source 401, and the inner and outer receive fibers are coupled to at least an inner and an outer channel of a multichannel spectrographic detector 600 as heretofore described. In an alternative embodiment, intended for direct contact with tissue, lenses 710, 712, are omitted, with other components remaining as herein described. In an alternative embodiment, a single-point probe 700 is positioned lens-uppermost below a transparent planar surface 714, and attached to a mechanical X-Y scanning apparatus 715. The probe 700 is mounted and scanned at an oblique angle relative to the planar surface 714 to reject specularly reflected light from entering the detection path. In this embodiment, a surgically-removed specimen 717, which may include part or all of a tumor 719, is positioned on the transparent planar surface 714, and scanning apparatus 715 draws probe 700 across surface 714. Light from the probe passes through the surface 714, while light scattered by specimen 717 and tumor 719 is received by probe 700 and admitted to probe receive fibers 704, 706, whence it is detected by spectrographic detectors 600. Image processing system 128 receives X-Y coordinate-pair information from scanning apparatus 715 and spectral information from detectors 600, executes the classifier on the spectral information for each coordinate pair, and uses tissue-classification produced by the classifier to construct a map of tissue classification of the tissue. This tissue-classification may is then displayed to the surgeon as previously described.

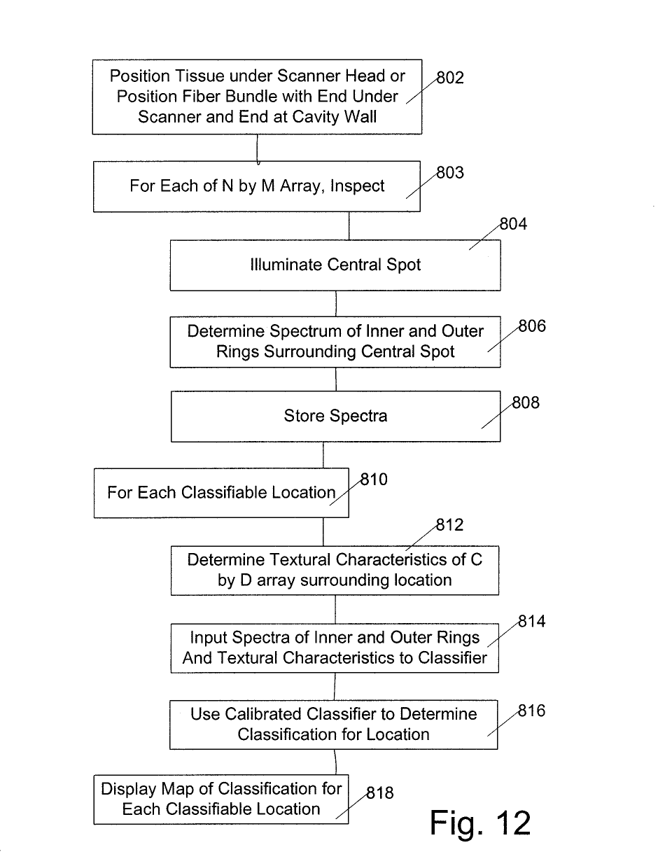

The scanning apparatus herein described operates according to FIG. 12. The surgeon begins a particular type of surgery in the normal way, identifies tumor, and removes a portion of tissue. The portion of removed tissue is positioned 802 on a stand under scanner head 503, or alternatively an end of the coherent fiber bundle is positioned under scanner head 503, with the other end of the bundle positioned at a suspect edge of the surgical cavity. For convenience, two scanner heads may be provided, one tissue-scanning head for scanning tissue on the stand, and one fiber-scanning head attached to the coherent fiber bundle.

The scanner then proceeds to inspect 803 tissue at an N by M array of locations on the tissue. In an embodiment, the scanner scans tissue at one-tenth-millimeter resolution in a 100 by 100 array (N and M being 100), or scans a scanner end of the fiber bundle with sufficient resolution that tissue adjacent the tissue end of the bundle is scanned at tenth-millimeter resolution or better. At each location, the tissue is illuminated 804 through the central fiber, light from the central fiber being focused on the tissue location, and spectra for both the inner and outer receive-fiber ring are determined 806 and stored 808 in memory of the image processing system. A small number of locations around the perimeter of the scanned locations are excluded from classifiable locations because full texture information for those locations is not available, if a surgeon wishes classification of those locations the tissue may be repositioned on the stand or the fiber bundle repositioned in the wound.

For each classifiable location, texture parameters are determined 812 from spectra in a C by D texture array surrounding the location to be classified, in a particular embodiment C and D are both five. In an embodiment this is done by summing spectral intensity for each location in the texture array to provide a gray level for each location, a C by D gray-level co-occurrence matrix (GLCM) representation is constructed, and texture parameters including spatial average, contrast, correlation and homogeneity parameters computed for the texture array.

The spectra from inner and outer rings, together with the texture parameters, are input 814 to a kNN classifier that has been provided with classification calibration parameters trained on tissue types expected to be encountered during the type of surgery being performed; calibration parameters for brain surgery will differ from those used for breast surgery. The classifier provides 816 a classification for each location, which is stored in memory.

Classifications for each classifiable location are then mapped from memory and displayed 818 as a map of tissue classifications for review by the surgeon. The surgeon may then remove additional tissue as needed to ensure adequate tumor margins

In an alternative embodiment 848, scan head 850 (FIG. 13) receives light from a first 401 and a second 401A light source operating under control of image processing system 128 via afferent fibers 852, 854 respectively. Scan head 850 has an inner ring of receive fibers 856 and an outer ring of receive fibers 858 coupled to individual channels of multichannel spectrographic detector 600. In a first, non-polarizing, mode, light from afferent fiber 852 passes through telecentric lens 860 and polarizing beamsplitter 862 to form an axial beam 864 scanned by scanner 866 through telecentric lens 868 onto tissue 870. Light scattered or reflected by tissue 870 is received from tissue by telecentric lens 868 as lateral beams 872 through polarizing beamsplitter 862 and lens 860 onto receive fibers 856, 858, whence the light is directed to detector 600 and processed as previously described to prepare a map of tissue classifications.

In a second, polarizing, mode, first light source 401 is turned off, and second light source 401A is turned on. Light from light source 401A passes from fiber 854 through polarizing beamsplitter 862 and polarized in a second direction to form an axial beam 864 scanned by scanner 866 through telecentric lens 868 onto tissue 870. Light scattered or reflected by tissue 870 is received from tissue by telecentric lens 868 as lateral beams 872 through polarizing beamsplitter 862, where light polarized in the second direction is diverted to absorber 874 and light polarized in the first direction passes through lens 860 onto receive fibers 856, 858, whence the light is directed to detector 600 and processed to prepare a second, or polarized, map of tissue classifications. In an embodiment, first and second polarizations are linear polarization states having orthogonal axes.

In an alternative embodiment, additional polarization optics are introduced in the place of polarizing beam splitter 862 to allow illumination and reception of orthogonal circular or elliptical polarization states. In an alternative embodiment having a conventional beamsplitter in place of polarizing beamsplitter 862—a transmit polarizing filter 863 may be mounted on a filter-rotating or filter-exchanging apparatus 865, such as a filter wheel and wheel-rotator, to permit polarizing and non-polarizing operation with a single light source, such as light source 401A, and a receive polarizing filter 867 is positioned in an optical path between scanner 866 and receive optical fibers 856, 858. In a particular embodiment, the filter-exchanging apparatus 865 has multiple polarizing filters, permitting the system to record spectra at each pixel for each polarization state provided by selected transmit filters 863. Further, light scattered once or only a few times may retain some residual polarization, so that by recording light spectra of each pixel at two or more polarizations of the same scan area this residual polarization of less-scattered light may be sensed, thereby permitting the system to derive tissue-type information primarily from polarization signatures of light scattered only once or a few times. In an alternative embodiment individual receive-fiber polarizing filters are provided at the lens 860 end of each receive fiber 856, 858. These receive-fiber polarizing filters are positioned such that the receive fiber filters are oriented in a rotating pattern of two, three, or four polarizations P1, P2, P3, and/or P4, in a pattern in each of the inner fibers 506 and outer receive fiber 514 rings, such that spectra obtained from fibers of each ring provide spectra of light received several fibers in each of the polarizations P1, P2, P3, and P4. Spectra of received light obtained in this way, together with the fact that light scattered once or only a few times may retain some residual polarization, permits the system to map select polarization properties of light scattered from the tissue and to use these polarization properties to derive tissue-type information primarily from light scattered only once or a few times.

In an alternative embodiment, two or more pre-defined polarization states are generated and analyzed in the same scan area to allow extraction and use of maps of select polarization properties of scattered light from tissue for classification. The combination of absorption insensitive-sampling and rejection of specularly reflected and multiply scattered light, allows extraction of polarization properties of the superficial tissue structures, which are otherwise lost.

In an embodiment, the scanning optics parameters, such as diameter of the scanning beam, focal length, effective NA, etc., are modified to permit operation with an optimized effective depth of focus.

In an alternative embodiment, a stimulus-wavelength-blocking receive filter adapted to pass a fluorescence emission wavelength is provided as part of receive polarizing filter 867. In this embodiment, a second transmit filter 863 of filter exchanging apparatus 865 is a stimulus-wavelength-passing filter for passing a fluorescence stimulus wavelength, a first transmit filter 863 is a clear filter, and a third transmit filter 863 is a polarizing filter. In this embodiment, a first spectra is captured for each pixel using the first, clear, transmit filter 863 to provide an unpolarized scatter image, a second spectra is captured for each pixel using the second, high-pass, filter to provide a fluorescence image, and the third, polarized, filter is captured for each pixel to provide a polarized image. All three images, fluorescence, unpolarized, and polarized, may then be provided to a surgeon or used by a classifier 124 in image processing system 128 to determine a tissue type for each pixel. In yet another embodiment, receive filter 867 is mounted on a filter-exchanging apparatus, thereby permitting imaging with additional alternatives of polarization and wavelength.

### Combinations of Elements

The components of the optical system, illumination system, optical fibers and bundles, detection system, and tissue classification system herein described may be utilized in a variety of combinations, some of which are described as follows:

A central-illumination scattering-based tissue-classifying system designated A including a plurality of optical fibers, each optical fiber having a first end and a second end; the first end of the optical fibers formed into a planar array; a broadband illuminator coupled to the second end of a source optical fiber of the optical fibers, wherein the plurality of optical fibers include a plurality of first receive optical fibers, the first end of the first receive optical fibers forming at least one ring around the first end of the source optical fiber, the second end of the first receive optical fibers coupled to at least a first channel of a spectrographic detection system; Apparatus configured to scan light from the source optical fiber across tissue; a processor coupled to receive data from the spectrographic detection system and having machine readable instructions for determining a classification of a type of tissue illuminated by the source fiber based upon spectra of light received from the tissue, and to provide a representation of tissue type distribution across the tissue. In most embodiments, including systems designated AB-AL the representation of tissue type distribution is an image or map of tissue types determined by repeatedly performing classification of the type of tissue as the light from the first end of the source optical fiber is scanned across the tissue.

A system designated AB including the system designated A and further including an optical system configured to focus light from the first end of the source optical fiber onto tissue, and light from the tissue onto the first end of the first receive optical fibers.

A system designated AC including the system designated AB wherein the optical system is adjustable to a plurality of predetermined magnification and/or demagnification settings.

A system designated AD including the system designated A or AB and wherein the scanning apparatus is configured to scan light from the first end of the source optical fiber across an area of tissue, wherein the processor is configured to determine spectra for an N by M array of classification locations determined as locations where light is provided from the first end of the source fiber to the tissue, and to store classifications determined therefrom in a, memory, and wherein the machine readable instructions further comprise instructions for mapping the classification of a type of tissue, where N and M are integers.

A system designated AE including the system designated AD, AC, AB, AA, or A wherein the plurality of optical fibers comprise a plurality of second receive optical fibers, the first end of the second receive optical fibers forming at least one ring around the first receive optical fibers, the second end of the second receive optical fibers coupled to at least a second channel of the spectrographic detection system.

A system designated AEA including the system designated AE, AD, AC, AB, or A wherein the optical system is configured to reject specular reflections from tissue using geometric separation or polarization discrimination.

A system designate AF including the system designated AE, AEA, AD, AC, AB, AA, or A wherein the machine readable instructions for determining a classification of a type of tissue at each classification location considers spectra acquired from at least the first and second receive optical fibers and textural information derived from data acquired at at least a C by D textural array of classification locations centered on the classification location, where C and D are integers.

A system designate AH including the system designated AE, AEA, AD, AC, AB, AA, or A wherein the machine readable instructions for determining a classification of a type of tissue at each classification location consider spectra acquired from at least the first receive optical fibers and textural information derived from data acquired at at least a C by D textural array of classification locations centered on the classification location, where C and D are integers.

A system designated AI including the system designated AF or AH wherein C and D are both five.

A system designated AJ including the system designate A, AA, AB, AC, AD, AE, AEA, AF, AH, or AI wherein the machine readable instructions for determining a classification of a type of tissue at each classification location comprise a classifier of the k-nearest-neighbors type.

A system designated AK including the system designated AEA, AF, AH, AI or AJ further including at least one polarizing device selected from the group consisting of a polarizing beamsplitter and at least one polarizing filter, the polarizing device disposed such that light focused from the source fiber onto the tissue has a first polarization, and light received into the detection system has a second polarization, the polarizing beamsplitter and polarizing filter configured to reject specular reflection from tissue.

A system designated AL including the system designated A, AA, AB, AC, AD, AE, AF, AH, AI or AJ further including a transmit stimulus-wavelength-passing filter and a receive stimulus-wavelength-blocking filter configured to pass fluorescent light from the tissue to the detection system.

A method designated B of classifying a type of tissue including illuminating a classification location on the tissue with a broad-spectrum light; capturing spectra of light received from at least an inner and an outer ring of tissue surrounding the illuminated location; using the captured spectra in an automatic classifier to determine a tissue type; scanning the classification location across a surface of the tissue, and preparing an image illustrating distribution of tissue type at the surface of the tissue.

A method designated BA including the method designated B further and including determining textural parameters from an array of locations surrounding the classification location, and using the textural parameters during the step of using the captured spectra in the automatic classifier to determine the tissue type.

A method designated BB including the method designated B or BA, wherein the automatic classifier is of the k-nearest-neighbor type and is provided with calibration data for tissue types likely to be encountered during a particular type of surgery.

A method designated BC including the method designated B, BA, or BB wherein the step of illuminating comprises illuminated with a light having a first polarization, and wherein the step of capturing spectra determines spectra of light having at least a second polarization different from the first polarization and thereby rejecting at least some light specularly reflected from the tissue.

A method designated BC including the method designated B, BA, or BB wherein the step of capturing spectra further determines spectra of light having at least a third polarization and thereby determining a polarization of light received from the tissue.

A method of treating a patient, wherein the tissue is tissue either of the patient, or tissue surgically removed from the patient, including the method designated BC, BB, BA, or B, and further including presenting the image illustrating distribution of tissue type to a surgeon, and, where possible, the surgeon using the image to select tissue for surgical removal.

A central-illumination scattering-based tissue-classifying system designated C including: a coherent bundle of optical fibers, the bundle having a first end and a second end, the second end configured for placement against tissue; a broadband illuminator coupled to illuminate a first region on the first end of the bundle; optics configured to collect light received from a first annular region surrounding the first region of the bundle into at least a first channel of a spectrographic detection system; apparatus configured to scan the first region and the first annular region across the first end of the bundle; a processor coupled to receive data from the spectrographic detection system and having machine readable instructions for determining a classification of a type of tissue illuminated by light from the second end of the bundle based upon spectra of light scattered by the tissue, and to provide a representation of tissue type distribution across the tissue.

A system designated CA including the system designated C and further including optics to collect light received from a second annular region surrounding the first annular region, and to direct that light into at least a second channel of the spectrographic detection system.

While the invention has been particularly shown and described with reference to particular embodiments thereof, it will be understood by those skilled in the art that various other changes in the form and details may be made without departing from the spirit and scope of the invention. It is to be understood that various changes may be made in adapting the invention to different embodiments without departing from the broader inventive concepts disclosed herein and comprehended by the claims that follow.

1-4. (canceled)

5. A central-illumination scattering-based tissue-classifying system comprising:
a plurality of optical fibers, each optical fiber having a first end and a second end;
the first end of the optical fibers formed into a planar array;
a broadband illuminator coupled to the second end of a source optical fiber of the optical fibers;
wherein the plurality of optical fibers comprise a plurality of first receive optical fibers, the first end of the first receive optical fibers forming at least one ring around the first end of the source optical fiber, the second end of the first receive optical fibers coupled to at least a first channel of a spectrographic detection system;
apparatus configured to scan light from the source optical fiber across tissue;
a processor coupled to receive data from the spectrographic detection system and having machine readable instructions for determining a classification of a type of tissue illuminated by the source fiber based upon spectra of light received from the tissue, and to provide a representation of tissue type distribution across the tissue;
an optical system configured to focus light from the first end of the source optical fiber onto tissue, and light from the tissue onto the first end of the first receive optical fibers;
wherein the processor is configured to determine spectra for an N by M array of classification locations and store classifications determined therefrom in a memory, and wherein the machine readable instructions further comprise instructions for mapping the classification of a type of tissue, where N and M are integers wherein the plurality of optical fibers comprise a plurality of second receive optical fibers, the first end of the second receive optical fibers forming at least one ring around the first receive optical fibers, the second end of the second receive optical fibers coupled to at least a second channel of the spectrographic detection system.

6. The system of claim 5 wherein the optical system is configured to reject specular reflections from tissue using geometric separation or polarization discrimination.

7. The system of claim 5, wherein the machine readable instructions for determining a classification of a type of tissue at each classification location considers spectra acquired from at least the first and second receive optical fibers and textural information derived from data acquired at at least a C by D textural array of classification locations centered on the classification location, where C and D are integers.

8. The system of claim 7 wherein C and D are both five.

9. The system of claim 5, wherein the machine readable instructions for determining a classification of a type of tissue at each classification location considers spectra acquired from at least the first receive optical fibers and textural information derived from data acquired at at least a C by D textural array of classification locations centered on the classification location, where C and D are integers.

10. The system of claim 9 wherein C and D are both five.

11. The system of claim 10 wherein the machine readable instructions for determining a classification of a type of tissue at each classification location comprise a classifier of the k-nearest-neighbors type.

12. The system of claim 5, further comprising at least one polarizing device selected from the group consisting of a polarizing beamsplitter and at least one polarizing filter, the polarizing device disposed such that light focused from the source fiber onto the tissue has a first polarization, and light received into the detection system has a second polarization, the optical system configured to reject light specularly reflected from tissue.

13. The system of claim 5, further comprising a transmit stimulus-wavelength-passing filter and a receive stimulus-wavelength-blocking filter configured to pass fluorescent light from the tissue to the detection system.

14. A method of classifying a type of tissue comprising:
illuminating a classification location on the tissue with a broad-spectrum light;
capturing spectra of light received from at least an inner and an outer ring of tissue surrounding the illuminated location;
using the captured spectra in an automatic classifier to determine a tissue type;
scanning the classification location across a surface of the tissue, and
preparing an image illustrating distribution of tissue type at the surface of the tissue.

15. The method of claim 14 further comprising:
determining textural and statistical parameters from an array of locations surrounding the classification location, and using the textural parameters during the step of using the captured spectra in the automatic classifier to determine the tissue type.

16. The method of claim 15 wherein the automatic classifier is of the k-nearest-neighbor type and is provided with calibration data for tissue types likely to be encountered during a particular type of surgery.

17. The method of claim 15, wherein the step of illuminating comprises illuminating with a light having a first polarization, and wherein the step of capturing spectra determines spectra of light having at least a second polarization different from the first polarization, thereby rejecting at least some light specularly reflected from the tissue.

18. The method of claim 17 wherein the step of capturing spectra further determines spectra of light having at least a third polarization thereby determining a polarization of light received from the tissue.

19. A central-illumination scattering-based tissue-classifying system comprising:
a coherent bundle of optical fibers, the bundle having a first end and a second end, the second end configured for placement against tissue;
a broadband illuminator coupled to illuminate a first region on the first end of the bundle;
optics to collect light received from a first annular region surrounding the first region of the bundle into at least a first channel of a spectrographic detection system;
apparatus configured to scan the first region and the first annular region across the first end of the bundle;
a processor coupled to receive data from the spectrographic detection system and having machine readable instructions for determining a classification of a type of tissue illuminated by the second end of the fiber bundle based upon spectra of light scattered by the tissue, and to provide a representation of tissue type distribution across the tissue.

20. The system of claim 19 further comprising optics to collect light received from a second annular region surrounding the first annular region, and to direct that light into at least a second channel of the spectrographic detection system.
